(19) United States
(12) Patent Application Publication
(10) Pub. No.: US 2014/0005643 A1
(43) Pub. Date: Jan. 2, 2014

(54) OPTICAL SCANNING DEVICE

(71) Applicant: METAL INDUSTRIES RESEARCH&DEVELOPMENT CENTRE, (US)

(72) Inventors: Yi-San CHANG, Tainan City (TW); Yu-Jen LU, Tainan City (TW); Yu-Sung HSIEH, Tainan City (TW); Shyang-Jye CHANG, New Taipei City (TW); Sheau-Chiou CHAO, Tainan City (TW)

(73) Assignee: METAL INDUSTRIES RESEARCH&DEVELOPMENT CENTRE, KAOHSIUNG (TW)

(21) Appl. No.: 13/724,322

(22) Filed: Dec. 21, 2012

(30) Foreign Application Priority Data
Jun. 27, 2012 (TW) 101123014

(51) Int. Cl. A61B 18/20; B23K 26/08; G02B 26/10

(52) U.S. Cl. CPC A61B 18/203; G02B 26/10; B23K 26/0807
USPC 606/9; 359/198.1; 219/121.8

(57) ABSTRACT

An optical scanning device is adapted to output a light beam, and includes a base, a transmission mechanism, a rotary power source and a holder. The transmission mechanism is disposed on the base. The rotary power source is adapted to generate a rotation and mechanically connected to the transmission mechanism so as to drive the transmission mechanism to generate the rotation. The holder is disposed to the transmission mechanism and includes a first lateral section, wherein when the transmission mechanism and the holder are synchronously rotated, the first lateral section also rotates the output light beam and thus the output light beam forms a light spot having a scanning area projected on a destination.

### CROSS-REFERENCE TO RELATED APPLICATION

This application claims the benefit of Taiwan Patent Application No. 101123014, filed on Jun. 27, 2012, which is hereby incorporated by reference for all purposes as if fully set forth herein.

### BACKGROUND OF THE INVENTION

1. Field of Invention

The present invention is relative to an optical scanning device, and more particularly, to an optical scanning device that utilises of a rotated mechanism to enlarge the scanning area.

2. Related Art

According to today's large-range laser skin treatment technology, doctors use handheld laser handpiece to apply the laser beam on patient's skin with their bare hands. If the surgery takes longer hours, the doctors may lose concentration and jeopardize the stability of the treatment quality. The instability might be caused either when the laser beam shoots an affected area for a long period of time to induce severe skin burn, or when doctors' shake of hands causes miss-shots of the laser beam and induce unnecessary damages to the normal skin. However, if doctors reduce the output energy density of the laser beam, either the time of treatment will increase, or the treatment will fail.

U.S. Pat. No. 5,860,967 discloses a laser treatment system named Dermatological Laser Treatment System with Electronic Visualization of The Area Being Treated. This treatment instrument allows doctors to directly position treatment area via a monitor and increase precision of locating treatment area via visual systems. The laser beam can be precisely controlled within the treatment area by means of a deflection manner. The laser treatment system disclosed by this prior art is a handheld laser micro surgical instrument for small-area treatment. It is primarily used to provide doctors with more precision and stableness during the treatment processes. However, a hand-held laser microsurgical instrument disclosed by this prior art cannot be applied to large-area treatments.

U.S. Pat. No. 7,441,899 discloses a device having a Panretinal Laser Fundus Contact Lens, which is helpful to automate the panretinal laser. A plate with a hole is located inside this device, and is used to install high-reflective mirrors or prisms that divert the laser beam. The center of the plate is provided with a mirror moved and driven by a micromotor, which turns the mirror to change the direction of the laser beam. In the bottom of this device is an annular mirror or a prism used to collect the laser beam and divert it to an inner of the eye. The idea of the prior art primarily utilizes a rotary mirror set to construct a treatment area. However, the prior art is primarily used in eye treatment, namely the device that is applied to small-area laser treatment.

In addition, typical large-area laser scan technique is not only used in human skin treatment, but also can be used as an annealing treatment apparatus in industry. TW Patent No. 1271805 discloses a laser annealing method and a laser annealing treatment apparatus, wherein the laser annealing treatment apparatus is acted as an uniformizing apparatus, which is constituted by a diffractive optical element or a combination of a Powell's lens and a cylindrical lens. However, the apparatus disclosed by the prior art requires complicated the combination of optical lens to transfer the laser from spots to a long and narrow beam.

Therefore, an optical scanning device is needed for solving the aforementioned problems.

### SUMMARY OF THE INVENTION

The purpose of the present invention is to overcome the dilemma that current optical scanning device can be only applied to scanning small areas. The dilemma also takes users tremendous amount of time when scanning large areas.

To achieve the foregoing object, the present invention provides an optical scanning device adapted to output a light beam, and including a base, a transmission mechanism, a rotary power source and a holder. The transmission mechanism is disposed on the base. The rotary power source is adapted to generate a rotation and mechanically connected to the transmission mechanism so as to drive the transmission mechanism to generate the rotation. The holder is disposed to the transmission mechanism and includes a first lateral section, wherein when the transmission mechanism and the holder are synchronously rotated, the first lateral section also rotates the output light beam and thus the output light beam forms a light spot having a scanning area projected on a destination.

The present invention provides the optical scanning device further comprising: an optical disposed above the base and adapted to generate the light beam; and an optical reflector set comprising a first reflector and a second reflector, wherein the first reflector is disposed on the transmission mechanism and located between the first lateral section and the second lateral section, and the second reflector is disposed on the first lateral section, whereby the light source emits the light beam to the first reflector, the first reflector then reflects the light beam onto the second reflector, and the second reflector reflects and further outputs the light beam to the destination.

The present invention provides the optical scanning device further comprising: a light source disposed on the first lateral section and is adapted to generate the light beam.

The light beam of the optical scanning device of the present invention primarily utilises the idea of “a rotated mechanism.” There are two rotating manners in the present invention: the first one is that the optical reflector set is rotated, and the second one is that the light source (such as Laser source) is rotated. These rotating manners both make the range projected from the outputted Laser beam that is larger than the size of the inputted Laser beam; meanwhen, the density of emitting energy will not decrease so there is no need to intentionally raise the Laser input energy. The additional benefit is to save the cost on energy usage and to lower the possibility of purchasing higher Laser energy equipment.

The following sections and figures will illustrate in detail the aforementioned and other purposes, features and benefits of the present invention:

### BRIEF DESCRIPTION OF THE DRAWINGS

FIG. 1 is a schematic perspective view of the optical scanning device in the first embodiment of the present invention;

FIG. 2 is a schematic top view of the adjustable diameter holder of the optical scanning device in the first embodiment of the present invention;

FIG. 3 is a schematic perspective view of the optical scanning device in the second embodiment of the present invention;

FIG. 4 is a schematic perspective view of the optical scanning device in the third embodiment of the present invention;

FIG. 5 is a schematic perspective view of the optical scanning device in the fourth embodiment of the present invention;

FIG. 6a˜6c are schematic lateral views of the optical scanning device in the first embodiment of the present invention, which respectively show the light beam deviating from, being parallel with, or moving toward a rotation center line of the optical scanning device and further being emitted to a destination;

FIG. 7a˜7c are schematic lateral views of the optical scanning device in the third embodiment of the present invention, which respectively show the light beam deviating from, being parallel with, or moving toward a rotation center line of the optical scanning device and further being emitted to a destination;

FIG. 8a˜8c are schematic top views of the optical scanning device in the present invention, which show a light spot having the first different scanning area emitted from the light beam; and

FIG. 9a˜9c are schematic top views of the optical scanning device in the present invention, which show a light spot having the second different scanning area emitted from the light beam.

### DETAILED DESCRIPTION OF THE INVENTION

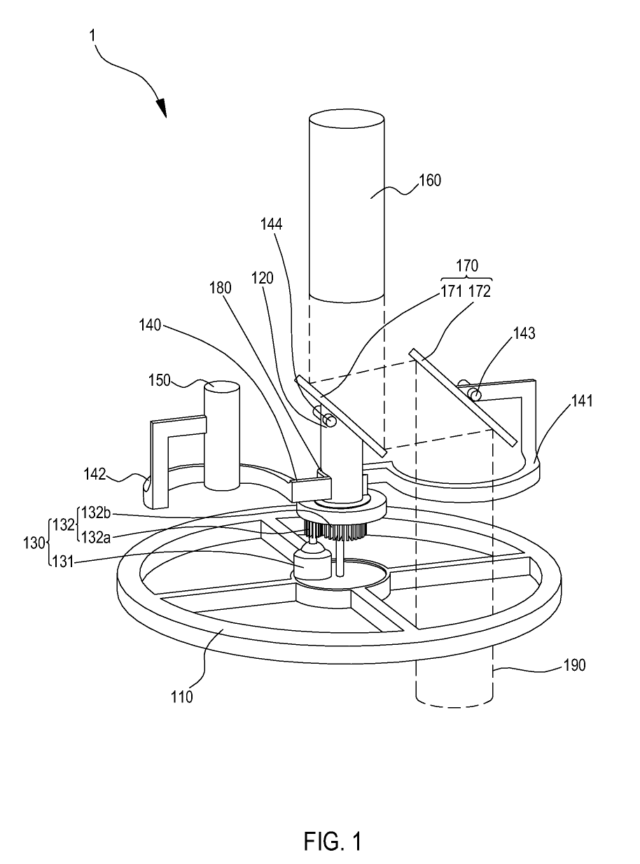

FIG. 1 is a schematic perspective view of an optical scanning device in the first embodiment of the present invention. The optical scanning device 1 is adapted to output a light beam 190, and includes a base 110, a transmission mechanism 120, a rotary power source 130, a holder 140, a poise 150, a light source 160 and an optical reflector set 170.

In the first embodiment of the present invention, the base 110 includes a ring and a cross holder is installed in the ring. The transmission mechanism 120 is disposed at the center of the cross holder.

The rotary power source 130, which is mechanically connected to the transmission mechanism 120, enables the transmission mechanism 120 to generate rotation. In the first embodiment of the present invention, in order to enable the rotary power source 130 to drive the transmission mechanism 120 and generate the rotation, the rotary power source 130 includes a motor 131 and a gear set 132. The motor 131 is disposed on the cross holder above the base 110, and the gear set 132 is disposed between the motor 131 and the transmission mechanism 120. The gear set 132 can consist of a first gear 132a and a second gear 132b. The first gear 132a of gear set 132 is dispose on the axle of the motor 131. The second gear 132b is disposed on the axle of the transmission mechanism 120 and engages with the first gear 132a. When the motor 131 rotates, the engaged relation between the first gear 132a and the second gear 132b will enable the motor 131 to drive the transmission mechanism 120.

The rotational speed of the transmission mechanism 120 can be controlled according to not only the rotational speed of the motor 131 but also the adjustment of the gear ratio between the first gear 132a and the second gear 132b. In another embodiment, the rotary power source 130 can utilize not only the motor 131 and the gear set 132 but also pneumatic rotation actuator device to generate rotation.

The light source 160, which is disposed above the base 110, is adapted to generate the light beam 190. The light source 160 can be any energy types of lights, such as Laser, Intense Pulsed Light, Light Emitting Diode (LED), or Infrared Ray (including Far Infrared Ray).

The holder 140 includes a first lateral section 141 and a second lateral section 142 opposite to the first lateral section 141. The holder 140 is disposed on the transmission mechanism 120. When the transmission mechanism 120 is driven to be rotated, the first lateral section 141 and second lateral section 142 of the holder 140 can be synchronously rotated; namely, the transmission mechanism 120 and the holder 140 both are rotated around the rotation center line of the optical scanning device 1.

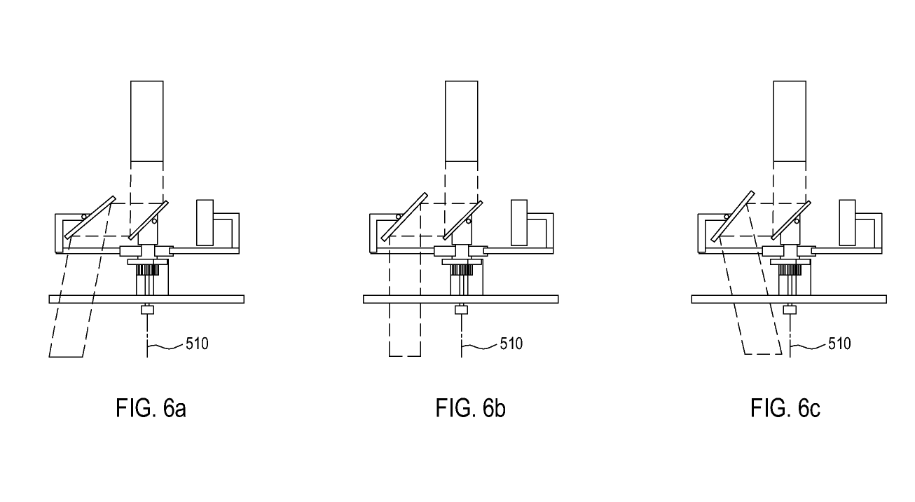

The optical reflector set 170 is used to reflect the light beam 190 emitted by the light source 160 and to further output the light beam 190 to the destination. In the first embodiment of the present invention, the optical reflector set 170 includes a first reflector 171 and a second reflector 172. The first reflector 171 is disposed on the transmission mechanism 120 (i.e. a position between the first lateral section 141 and the second lateral section 142 of the holder 140) and is rotated synchronously with the transmission mechanism 120. In addition, there can be an angle, for example, 45 degree angle, between the first reflector 171 and the emitting direction of the light source 160. Also, the second reflector 172 is disposed on the first lateral section 141 of the holder 140 and can be parallel with the first reflector 171. The light beam 190 of the light source 160 can emit onto the first reflector 171, and the first reflector 171 reflects the light beam 190 onto the second reflector 172. The second reflector 172 then reflects the light beam 190 and further outputs it to the destination. The outputted light beam 190 is parallel with the input light beam 190, and the output light beam 190 is also parallel with the rotation center line of the optical scanning device 1 (as shown in FIG. 6b). When the transmission mechanism 120 and the first lateral section 141 of the holder 140 are synchronously driven to be rotated, the first lateral section 141 also rotates the outputted light beam 190 at the same time (i.e. the first lateral section 141 rotates the light beam 190 reflected from the second reflector 172). Thus, the rotated and outputted light beam 190 forms a light spot that has a scanning area projected on a destination. The optical scanning device 1 of the present invention enlarges the scanning area and further enhances the heating efficiency of the light beam 190 by rotating the outputted light beam 190.

The poise 150 is disposed on the second lateral section 142 of the holder 140 and is utilized to maintain the stable balance of the holder 140 when the holder 140 is rotated.

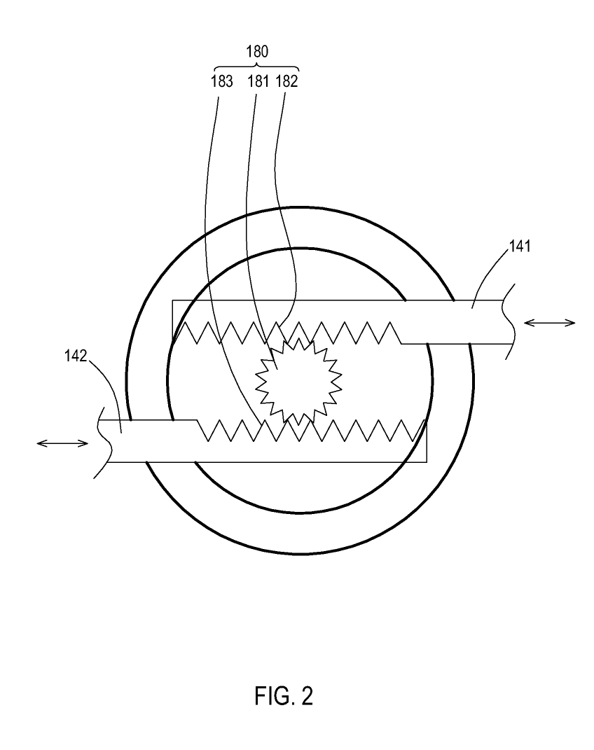

An adjustable diameter mechanism 180 is disposed between the first lateral section 141 and the second lateral section 142 of the holder 140, and is used to control the distance between the first lateral section 141 and the second lateral section 142. As shown in FIG. 2, the adjustable diameter mechanism 180 includes an adjustable diameter gear 181, a first gear tooth 182 and a second gear tooth 183. The adjustable diameter gear 181 is disposed between the first lateral section 141 and the second lateral section 142 of the holder 140. The first gear tooth 182 is physically connected to the first lateral section 141, and the second gear tooth 183 is physically connected to the second lateral section 142. The first gear tooth 182 and the second gear tooth 183 are respectively located at the both ends of the adjustable diameter gear 181, and engage with the adjustable diameter gear 181. Being driven by the adjustable diameter gear 181, the second lateral section 142 of the holder 140 will move outward when the first lateral section 141 of the holder 140 moves outward. This movement can thus maintain the stable balance of the holder 140 when the holder 140 is rotated.

In order to control the angel of the optical reflector 170 and then control the direction of the outputted light beam 190, a first angle adjuster 143 can be disposed between the second reflector 172 and the first lateral section 141 of the holder 140, and a second angel adjuster 144 can be disposed between the transmission mechanism 120 and the first reflector 171. For example, when a user needs a light spot having larger scanning area of the outputted light beam 190, the user can adjust the first adjuster 143 to deviate the light beam 190 from the rotation center line 510 of the optical scanning device 1 and to further emit the light beam 190 to the destination (as shown in FIG. 6a). When the user needs a light spot having smaller scanning area of the outputted light beam 190, the user can also adjust the first adjuster 143 to make the light beam 190 emitted on the second reflector 172 move toward the rotation center line 510 of the optical scanning device 1 and thus emit the light beam 190 to the destination (as shown in FIG. 6c).

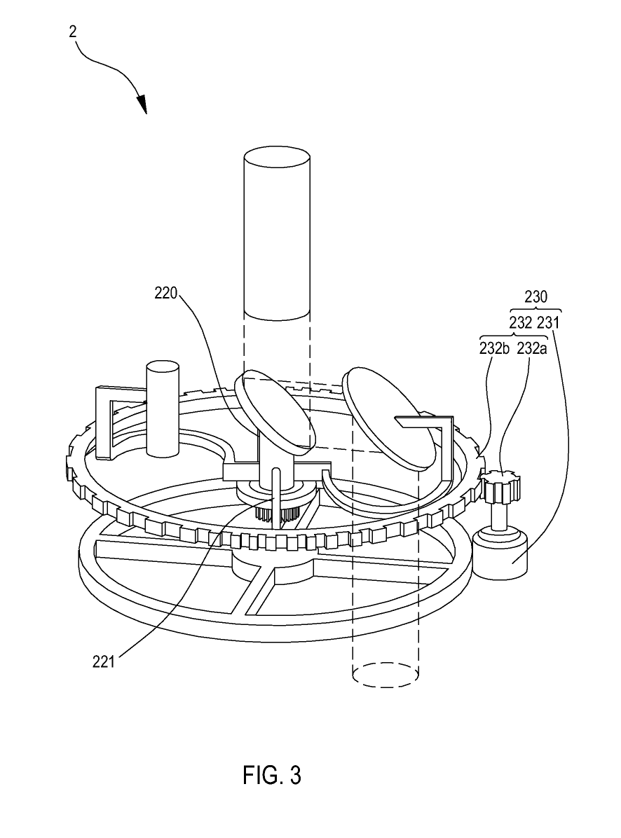

FIG. 3 is a schematic perspective view of an optical scanning device in the second embodiment of the present invention. The optical scanning device 2 in the second embodiment is substantially similar to the optical scanning device 1 in the first embodiment. The differences between the optical scanning device 1 and the optical scanning device 2 are: the components of the rotary power source 230 and their locations. In the second embodiment, in order to enable the rotary power source 230 and further drive the transmission mechanism 220 to generate a rotation, the rotary power source 230 includes of a gear set 232 and a motor 231. The gear set 232 consists of a first gear 232a and a second gear 232b. It should be particularly mentioned that the second gear 232b is a ring gear. In addition, the transmission mechanism 220 further includes a supporting holder 221, which is physically connected to the ring gear 232b. The ring gear 232b surrounds the periphery of the transmission mechanism 220 and is physically connected to the supporting holder 221 of the transmission mechanism 220. Or, the ring gear 232b surrounds a periphery of the transmission mechanism 220 and is physically connected to the holder 240. The motor 231 is mechanically connected to the ring gear 232b. The first gear 232a is physically connected to the axle of the motor 231 and engages with the ring gear 232b. When the motor 231 rotates, the first gear 232a can drive the ring gear 232b to be rotated and further drive the transmission mechanism 220 to generate the rotation of transmission mechanism 220 by means of the connection between the ring gear 232b and the supporting holder 221.

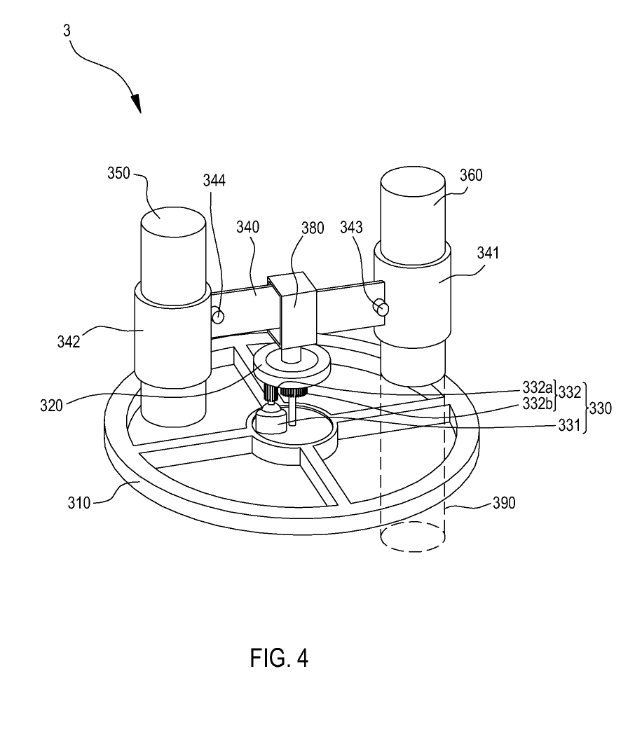

FIG. 4 is a schematic perspective view of an optical scanning device 3 in the third embodiment of the present invention. The optical scanning device 3 is adapted to output a light beam 390 and includes: a base 310, a transmission mechanism 320, a rotary power source 330, a holder 340, a poise 350 and a light source 360.

In the third embodiment of the present invention, the base 310 includes a ring, and a cross holder is installed in the ring. The transmission mechanism 320 is disposed at the center of the cross holder.

The rotary power source 330, which is mechanically connected to the transmission mechanism 320, drives the transmission mechanism 320 to generate rotation. In the third embodiment of the present invention, in order to enable the rotary power source 330 to drive the transmission mechanism 320 to be rotated, the rotary power source 330 includes a motor 331 and a gear set 332. The motor 331 is disposed on the cross holder above the base 310, and the gear set 332 is disposed between the motor 331 and transmission mechanism 320. The gear set 332 consists of a first gear 332a and a second gear 332b. The first gear 332a of the gear set 332 is disposed on the axle of the motor 331. The second gear 332b is disposed on the axle of the transmission mechanism 320 and engages with the first gear 332a, whereby the motor 331 drives the transmission mechanism 320 to be rotated.

The rotational speed of the transmission mechanism 320 can be controlled according to not only the rotational speed of the motor 331 but also the adjustment of the gear ratio between the first gear 332a and the second gear 332b. In another embodiment, the rotary power source 330 can utilise not only the motor 331 and the gear set 332 but also pneumatic rotation actuator device to generate rotation.

The holder 340 includes a first lateral section 341 and a second lateral section 342 opposite to the first lateral section 341. The holder 340 is disposed on the transmission mechanism 320. When the transmission mechanism 320 is driven to be rotated, the first lateral section 341 and the second lateral section 342 of the holder 140 can be rotated synchronously; namely, the transmission mechanism 320 and the holder 340 both are rotated around the rotation center line of the optical scanning device 3.

The light source 360, which is disposed on the first lateral section 341 of the holder 340, is used to generate the light beam 390. The light source 360 can be any energy types of lights, such as Laser, Intense Pulsed Light, Light Emitting Diode (LED), or Infrared Ray (including Far Infrared Ray)

The poise 350 is disposed on the second lateral section 342 of the holder 340 and is utilised to maintain the stable balance of the holder 340 when the holder 340 is driven to be rotated.

An adjustable diameter mechanism 380 disposed between the first lateral section 341 and the second lateral section 342 of the holder 340 is used to control the distance between the first lateral section 341 and the second lateral section 342. The structure and function of the An adjustable diameter mechanism 380 is not described repeatedly here. Please refer to the first embodiment and the FIG. 2.

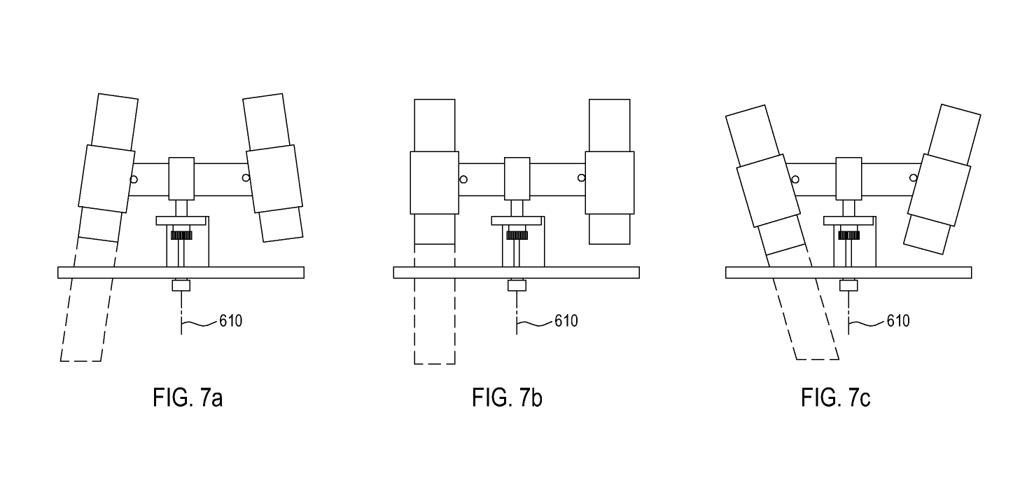

A first angle adjuster 343 can be disposed on the first lateral section 341 of the holder 340, and a second angle adjuster 344 can be disposed on the second lateral section 342 of the holder 340. The first angle adjuster 343 is used to control the emitting angle of the light beam 390 generated by the light source 360. When a user needs a light spot having larger scanning area of the outputted light beam 390, the user can adjust the first adjuster 343 to deviate the light beam 390 from the rotation center line 610 of the optical scanning device 3 and to further emit the light beam 390 to the destination (as shown in FIG. 7a). When the user needs a light spot having smaller scanning area of the outputted light beam, the user can also adjust the first adjuster 343 to emit the light beam 390 toward the rotation center line 610 of the optical scanning device 3 and to further emit the light beam 390 to the destination (as shown in FIG. 7c). The second angle adjuster 344 is used to control the angle of the poise 350. The angle of the second angle adjuster 344 will be adjusted accordingly when the angle of the first angle adjuster 343 is adjusted.

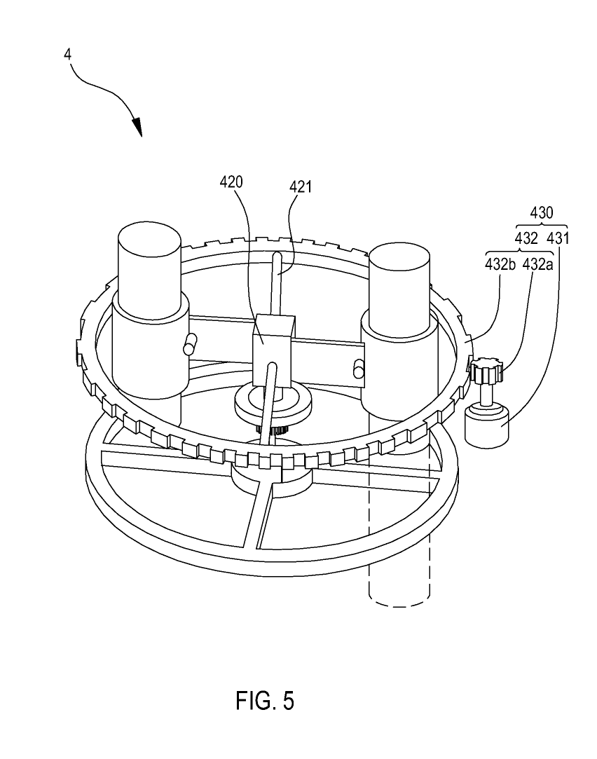

FIG. 5 is a schematic perspective view of the optical scanning device, which is the fourth embodiment of the present invention. The optical scanning device 4 in the fourth embodiment is substantially similar to the optical scanning device 3 in the third embodiment. The differences between the optical scanning device 4 in the fourth embodiment and the optical scanning device 3 in the third embodiment are: the components of the rotary power source 430 and their locations. In the fourth embodiment of present invention, in order to enable the rotary power source 430 to generate rotation and further drive the transmission mechanism 420, the rotary power source 430 includes a gear set 432 and a motor 431. The gear set 432 consists of a first gear 432a and a second gear. It should be particularly mentioned that the second gear is a ring gear 432b. In addition, the transmission mechanism 420 further includes a holder 421, which is physically connected to the ring gear 432b. The ring gear 432b surrounds the periphery of the transmission mechanism 420 and is physically connected to the supporting holder 421 of the transmission mechanism 420. Or, the ring gear 432b surrounds a periphery of the transmission mechanism 420 and is physically connected to the holder 440. The motor 431 is mechanically connected to the ring gear 432b. The first gear 432a is physically connected to the axle of the motor 431 and engages with the ring gear 432b. When the motor 431 rotates, the first gear 432a can drive the ring gear 432b to be rotated and further drive the transmission mechanism 420 to generate rotation by means of the connection between the ring gear 432b and the supporting holder 421.

FIG. 6a˜6c are the lateral views of the optical scanning device in the first embodiment of the present invention, which respectively show the outputted light beam deviating from, being parallel with, or moving toward a rotation center line of the optical scanning device and further being emitted to a destination. As shown in FIG. 6a, when users need a light spot having a larger scanning area of the outputted light beam, they can adjust the angle of the second reflector to deviate the light beam from the rotation center line 510 of the optical scanning device and to emit the light beam onto the destination. As shown in FIG. 6b, when users need a light spot having a middle scanning area of the outputted light beam, they can adjust the angle of the reflector to make the light beam parallel with the rotation center line 510 of the optical scanning device and to further emit the light beam to the destination. As shown in FIG. 6c, when users need a light spot having a smaller scanning area of the outputted light beam, they can adjust the angle of the reflector and emit the outputted light beam toward the rotation center line 510 of the optical scanning device and further emit the light beam to the destination.

FIG. 7a˜7c are the lateral views of the optical scanning device in the third embodiment of the present invention, which respectively show the light beam deviating from, being parallel with, or moving toward a rotation center line of the optical scanning device and further being emitted to a destination. As shown in FIG. 7a, when users need a light spot having a larger scanning area of the outputted light beam, they can adjust the angle of the light source to deviate the outputted light beam from the rotation center line 610 of the optical scanning device and emit the light beam to the destination. As shown in FIG. 7b, when users need a light spot having a middle scanning area of the outputted light beam, they can adjust the angle of the light source to make the light beam parallel with the rotation center line 610 of the optical scanning device and to further emit the light beam to the destination. As shown in FIG. 7c, when users need a light spot having a smaller scanning area of the outputted light beam, they can adjust the angle of the light source and emit the outputted light beam toward the rotation center line 610 of the optical scanning device and further emit the light beam to the destination.

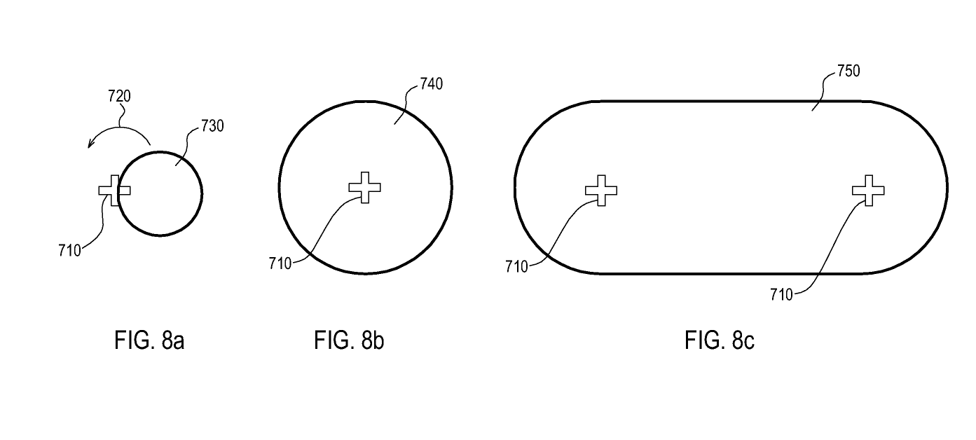

FIG. 8a˜8c are the top views of the optical scanning device of the present invention, which show light spots having the first different scanning area of the light beam. Assuming that the optical scanning device is set as the condition shown in the FIG. 6c or FIG. 7c, and the cross represents the rotation center line 710 of the optical scanning device. When the transmission mechanism does not be rotated, the light spot 730 having the scanning area of the outputted light beam will be a small circular spot, and the outputted light beam will be emitted toward the rotation center line 710 (as shown in FIG. 8a). When the transmission mechanism is rotated, the outputted light beam will move along the rotation direction 720 and form the light spot 740 having a larger circular scanning area as a solid circle shape (as shown in FIG. 8b). When the whole optical scanning device is moved along a straight line, the light spot 750 having the scanning area of the outputted light beam is shown in FIG. 8c.

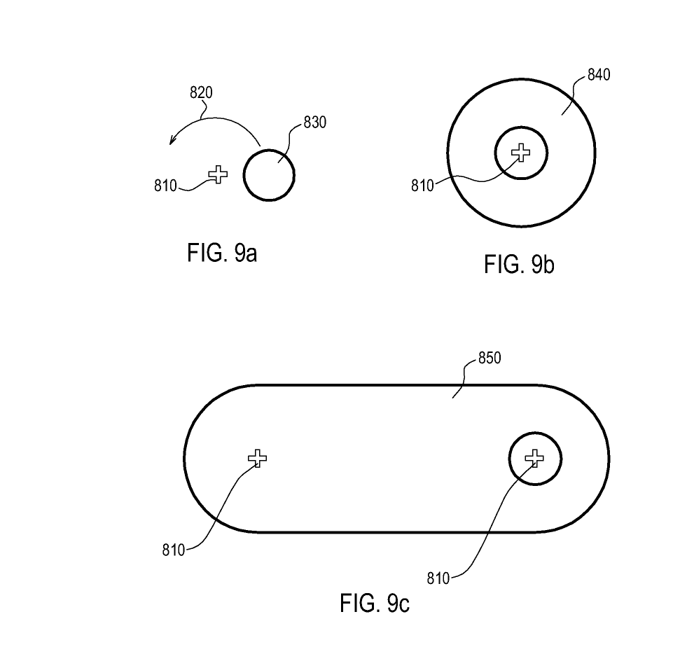

FIG. 9a˜9c are the top views of the optical scanning device of the present invention, which show light spot having the second different scanning area of the light beam. Assuming that the optical scanning device is set as the condition shown in the FIGS. 6a, 6b or FIGS. 7a, 7b, and the cross represents the rotation center line 810 of the optical scanning device. When the transmission mechanism does not be rotated, the light spot 830 having the scanning area of the outputted light beam will be a small circular spot, and the outputted light beam will deviate from or parallel the rotation center line 810 (as shown in FIG. 9a). When the transmission mechanism is rotated, the outputted light beam will move along the rotation direction 820 and form the light spot 840 having a larger annular scanning area; however, the rotation center line 810 is not projected by the light beam and thus the light spot 840 having a donut shape is formed (as shown in FIG. 9b). When the whole optical scanning device is moved along a straight line, the light spot 850 having the scanning area of the outputted light beam is shown in FIG. 9c.

As a result, by rotating the outputted light beam or the reflector, the optical scanning device of the present invention enlarges the light spot having the scanning area of the light beam and further enhances the efficiency for the heating of the light beam on the relevant application (For example: Medical Cosmetology Treatment or Thermal Annealing Treatment).

As applied in the Medical Cosmetology Treatment, the optical scanning device of the present invention, by emitting the light beam (such as Laser) on the skin (if the application can enlarge the scanning area and light beam can be still remained at the same energy density, thus it can further enhance the efficiency of the treatment and reduce the cost of the Laser hardware!), heats the collagen tissue in the dermis without causing any pain due to the heat accumulation at the epidermis. This optical scanning device can facilitate the generation, continuous reorganization and contraction of collagen and further results in firming skin.

As applied in the thermal annealing treatment, the optical scanning device of the present invention emits the light beam (such as Laser) on a material and facilitates the condition of crystalline grains change or structure change of grains, which is a type of intrinsic change in the material. The relative applications in the current market include: changing the intrinsic structure of si-based thin film used in Flat panel display, changing the intrinsic structure of the thin film solar cells and the changing the intrinsic structure of metal materials, etc.

The light beam of the optical scanning device of the present invention primarily utilises the idea of “a rotated mechanism.” There are two rotating manners in the present invention: the first one is that the optical reflector set is rotated, and the second one is that the light source (such as Laser source) is rotated. These rotating manners both make the range projected from the outputted Laser beam that is larger than the size of the inputted Laser beam; meanwhen, the density of emitting energy will not decrease so there is no need to intentionally raise the Laser input energy. The additional benefit is to save the cost on energy usage and to lower the possibility of purchasing higher Laser energy equipment.

In conclusion, the abovementioned demonstrates but does not limit the applications or embodiments of the present invention on the techniques utilised to solve problems. It is to be noted that various changes and modifications possibly relative to the present invention or such changes and modifications as being practiced within the scope of the present invention are intended to be encompassed by the present disclosure.

What is claimed is:

1. An optical scanning device adapted to output a light beam, the optical scanning device comprising:
a base;
a transmission mechanism disposed on the base;
a rotary power source adapted to generate a rotation and mechanically connected to the transmission mechanism so as to drive the transmission mechanism to generate the rotation; and
a holder disposed to the transmission mechanism and comprising a first lateral section;
wherein when the transmission mechanism and the holder are synchronously rotated, the first lateral section also rotates the outputted light beam and thus the outputted light beam forms a light spot having a scanning area projected on a destination.

2. The optical scanning device according to claim 1, further comprising:
a poise, wherein the holder further comprises a second lateral section which is opposite to the first lateral section, the poise is disposed on the second lateral section of the holder for maintaining a stable balance of the holder when the holder is rotated.

3. The optical scanning device according to claim 1, further comprising:
a light source disposed above the base and adapted to generate the light beam; and
an optical reflector set comprising a first reflector and a second reflector, wherein the first reflector is disposed on the transmission mechanism and located between the first lateral section and the second lateral section, and the second reflector is disposed on the first lateral section, whereby the light source emits the light beam to the first reflector, the first reflector then reflects the light beam onto the second reflector, and the second reflector reflects and further outputs the light beam to the destination.

4. The optical scanning device according to claim 3, wherein the holder further comprises an angle adjustor disposed between the first lateral section and the second reflector so as to control the direction of the outputted light beam.

5. The optical scanning device according to claim 1, further comprising a light source disposed on the first lateral section and is adapted to generate the light beam.

6. The optical scanning device according to claim 5, wherein the holder further comprises an angle adjustor disposed between the first lateral section and the light source so as to control the direction of the outputted light beam.

7. The optical scanning device according to claim 6, wherein the light beam deviates from, is parallel with, or moves toward a rotation center line of the optical scanning device and is further emitted to the destination.

8. The optical scanning device according to claim 6, wherein the light beam deviates from, is parallel with, or moves toward a rotation center line of the optical scanning device and is further emitted to the destination.

9. The optical scanning device according to claim 1, wherein the holder further comprises an adjustable diameter mechanism disposed between the first lateral section and the second lateral section and adapted to control the distance between the first lateral section and the second lateral section.

10. The optical scanning device as recited in claim 1, wherein the rotary power source comprises:
a motor disposed on the base; and
a gear set disposed between the motor and the transmission mechanism, whereby when the motor rotates, the engaged relation between the first gear and the second gear enables the motor to drive the transmission mechanism to generate a rotation.

11. The optical scanning device according to claim 1, wherein the rotary power source comprises:
a ring gear surrounding a periphery of the transmission mechanism and physically connected to the holder; and
a motor mechanically connected to the ring gear.

12. The optical scanning device according to claim 1, wherein the optical scanning device is applied in a Medical Cosmetology Treatment, the light beam is a Laser beam, the destination is a skin, and the light beam is adapted to be emitted on the skin.

13. The optical scanning device according to claim 1, wherein the optical scanning device is applied in a thermal annealing treatment, the light beam is a Laser beam, the destination is a material, and the light beam is adapted to be emitted on the material.
